(19) United States
(12) Patent Application Publication
(10) Pub. No.: US 2010/0239723 A1
(43) Pub. Date: Sep. 23, 2010

(54) SYSTEM AND METHOD OF TEMPERATURE ADJUSTMENT AND CONTROL OF FOOD PROCESSING/DISPENSING SYSTEM OR APPARATUS

(76) Inventors: Sean A. Pendergast, Berlin, MA (US); Steven Moysey, South Grafton, MA (US)

Correspondence Address: CESARI AND MCKENNA, LLP, 88 BLACK FALCON AVENUE, BOSTON, MA, 02210, US

(21) Appl. No.: 12/712,508

(22) Filed: Feb. 25, 2010

Related U.S. Application Data
(60) Provisional application No. 61/155,378, filed on Feb. 25, 2009.

(51) Int. Cl. G01N 33/02

(52) U.S. Cl. 426/231

(57) ABSTRACT

The invention is directed to improvements to the temperature control systems and methods used within a food processing and dispensing device. Specifically, the present invention measures, by a thermocouple assembly, the actual temperature of food product ingredients and transmits one or more signals to a CPU. The signals represent the actual temperature of the one or more food product ingredients measured by the first thermocouple assembly. Then the CPU employs one or more look-up tables within the CPU to determine a set point temperature of a food processing surface. The system and/or method measures the actual temperature of the food processing surface, using a second thermocouple assembly, and transmits one or more signals to the CPU representing the actual temperature of the food processing surface. The temperature of the food processing surface is then dynamically adjusted to the determined set point to offset the temperature of the food product ingredients.

### CROSS-REFERENCE TO RELATED APPLICATIONS

The present application claims the benefit of U.S. Provisional Patent Application Ser. No. 61/155,378, which was filed on Feb. 25, 2009, by Sean A Pendergast for a SYSTEM AND METHOD OF TEMPERATURE ADJUSTMENT AND CONTROL OF FOOD PROCESSING/DISPENSING SYSTEM OR APPARATUS and is hereby incorporated by reference.

### BACKGROUND OF THE INVENTION

1. Field of the Invention

The invention relates generally a system and method of temperature adjustment and control of a food processing and dispensing systems.

2. Background Information

Apparatuses and methods described in the following commonly owned copending patents and patent applications: U.S. Pat. Nos. 5,292,030, 5,433,967, 5,473,909, 5,603,257, 5,727,713, 5,758,571, 5,868,065, 6,698,228, 6,745,595, 6,907,741, 6,941,858, 6,952,928, 7,052,728, and 7,131,279; U.S. Patent Publication Nos: 2006/0054614, 2006/0162348, 2006/0162347, 2006/0003065, 2007/0251260; and PCT Application Nos.: WO 92/02146, WO 03/041513, WO 04/019707, and WO 06/076733, produce and dispense chilled and at least partially frozen food products, such as ice cream, frozen yoghurt and slushes. These food products are made fresh and on-demand with the apparatuses and methods disclosed in the above noted applications and patents. The above referenced devices produce frozen foods made to customer-based selections and specifications. Specifically, the apparatuses and methods receive a customer's selection/specification at a CPU from a user interface. The customer's selection/specification can include the type of base mix, such as, for example, a base mix for premium, low-fat or sugar-less ice cream, the desired flavorings, such as, for example, vanilla, chocolate, strawberry, etc. and the desired mix-ins to be added to the ice cream. In such apparatuses, base mixes may be stored at room temperature in a liquid and/or dry form, e.g., powder, for processing and chilling with one or more customer-selected flavorings and/or other additives. Base mixes, such as liquid base mixes, may be preferably stored within any of the apparatuses at temperatures ranging between from about 35° F. to about 40° F., requiring substantially less refrigeration than is required for frozen base mixes. Alternatively, or additionally, base mixes in liquid or powder form may be stored within one or more containers within any of the apparatuses at ambient or room temperatures, until an apparatus uses the base mix for production of a frozen food product in accordance with a customer's selection.

After a customer selects a base mix and one or more flavorings or other additives, is the above apparatuses combine or blend the ingredients, optionally, aerate the blended ingredients or product mix, and deposit the product mix on a processing plate or table for further processing such as, for example, chilling or at least partially freezing, to produce a final food product.

Thus, when the blended ingredients are initially deposited on the processing plate or table, they are still at or slightly below room temperature. Thus, the temperature of the processing surface is directed impacted. This can adversely impact the product quality of the ice cream being produced.

### SUMMARY OF THE INVENTION

The invention is directed to improvements to the temperature control systems and methods used within the above noted apparatuses and methods in order to accommodate and offset temperatures of any base mixes and/or flavorings deposited on the processing plate surface. More particularly, the invention provides a system and method of adjusting temperatures of a processing surface or table used to process, e.g., chill or at least partially freeze, a base mix, a flavoring and/or a product mix containing the base mix and/or the flavoring in order to offset the temperatures of the base mix and/or flavoring. As noted above, a base mix or flavoring may be stored at ambient or room temperatures within any of the above noted apparatuses. In addition, a base mix or flavoring may be loaded into any of these apparatuses at ambient or room temperatures.

The invention enables the apparatuses and methods to use a base mix or flavoring without the downtime required for the base mix or flavoring to refrigerate and cool to optimal or preferred temperatures, such as, for example, from about 35° F. to about 40° F. The invention improves any of the above noted apparatus and methods in that such apparatuses and methods produce a consistent food product from one customer order to another without significant downtime and with relatively consistent product qualities.

### BRIEF DESCRIPTION OF THE DRAWINGS

The invention description below refers to the accompanying drawings, of which:

FIG. 1 is a schematic diagram of the illustrative embodiment of the present invention including a temperature control system operatively coupled with a processing surface or table of a food processing system or apparatus;

FIG. 2 is a perspective view of a food module including the processing surface or table shown in FIG. 1;

FIG. 3 is an exploded perspective view of components of the processing surface or table shown in FIGS. 1 and 2;

FIG. 4 is a schematic graph illustrating a temperature scheme with various temperature values of the processing surface or table shown in FIGS. 1-3 correlated with temperature values of food product ingredient(s) or intermediate(s);

FIG. 5 is a flow diagram of one aspect of the invention including a method of temperature adjustment and control; and

### DETAILED DESCRIPTION OF AN ILLUSTRATIVE EMBODIMENT

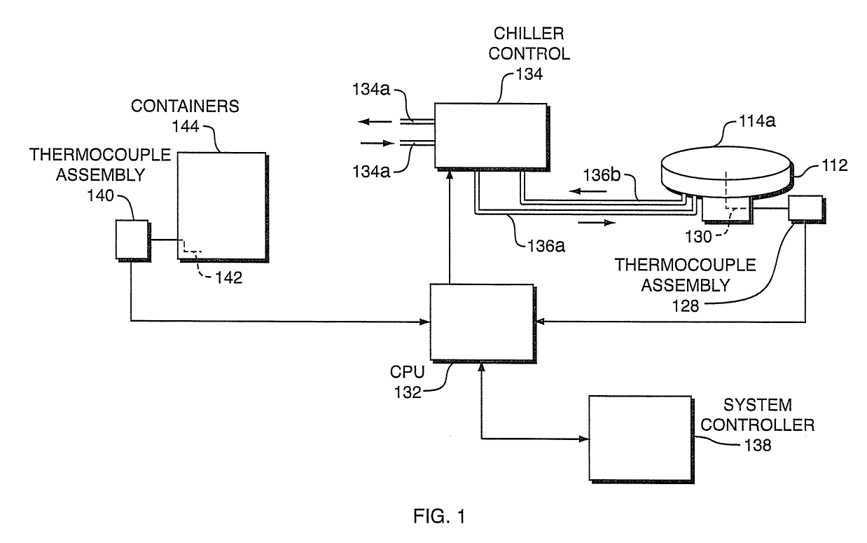

FIG. 1 is an illustrative embodiment of a temperature adjustment and control system 100 for adjusting and controlling temperatures of a food-processing surface or table 112, and/or other assemblies of a food processing system or apparatus in contact with food product ingredients and intermediates. The system 100 helps to accommodate temperatures of food product ingredients and intermediates when, for instance, such ingredients and intermediates are introduced into and/or stored within the food processing system or apparatus at ambient or room temperature.

More particularly, the system 100 of the invention helps to accommodate or offset the temperatures of food product ingredients and intermediates in order for a food processing system or apparatus to process such ingredients and intermediates at the required or optimal temperatures to produce a food product with certain required or desired properties and qualities. For purposes of describing the invention, the system 100 and the food processing surface or plate 112, and other assemblies in contact with food ingredients and intermediates, is described with reference to a system or apparatus for processing and dispensing a chilled or at least partially frozen food product, such as ice cream and frozen yoghurt. The system 100 however is not so limited and may incorporate into or integrate with a system or apparatus for processing and, optionally, dispensing any of a variety of food products. In addition, the invention is not limited to producing ice cream or frozen yoghurt, nor is limited for use in a system or apparatus that produces ice cream and frozen yoghurt, and envisions that the system 100 may be used to help to adjust and control temperatures in other food production systems and apparatuses.

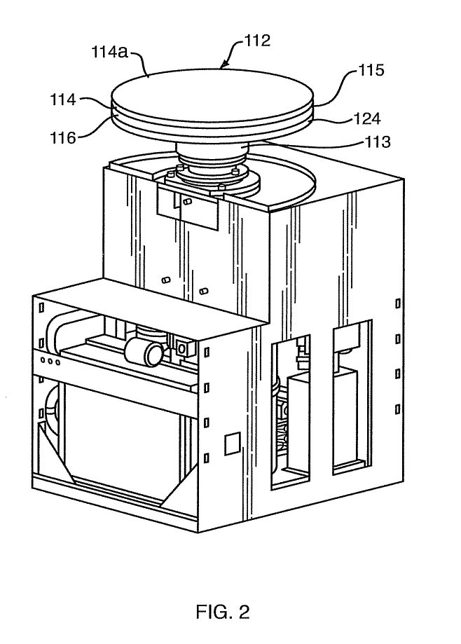

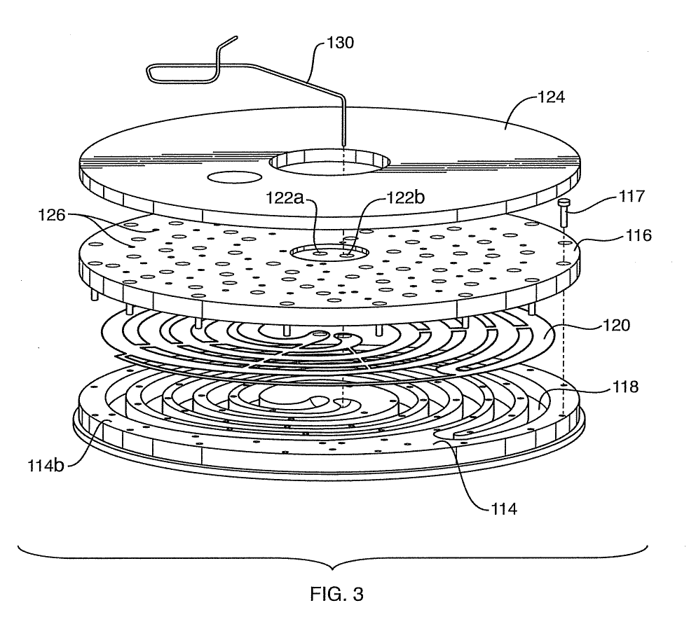

Referring to FIGS. 2 and 3, with further reference to FIG. 1, the system 100 includes the food-processing surface or table 112 having a central axis 113 and a periphery 115. As shown in FIGS. 2 and 3, the table 112 includes a first upper plate 114 and a second lower plate 116. A first face of the upper plate 114 forms a processing surface 114A, e.g., a chilling or freezing surface, of the table 112 that receives food product ingredients and mixtures thereof, all referred to hereinafter as a “product mix,” for chilling or at least partially freezing. A second face 114B of the upper plate 114 includes or defines a refrigerant channel 118 configured and operative to pass refrigerant fluid. The lower plate 116 and the upper plate 114 couple, e.g., via a number of screws or other fasteners 117, and a first face (not shown) of the lower plate 116 seals the refrigerant channel 118. A gasket 120 is disposed between the upper and the lower plates 114 and 116 to help seal the plates 114 and 116 and/or to help to reduce cross flow of refrigerant disposed in the channel 118. The coupled upper and lower plates 114 and 116 help to define the channel 118 that includes at least one entrance orifice 122A and at least one exit orifice 122B. A number of screws adjacent to both sides of the refrigerant channel 118 helps to maintain the channel 118 and facilitates the function of the gasket 120.

The plates 114 and 116 and the gasket 120 help to create the channel 118 including refrigerant passages, e.g., defined in a serpentine pattern, that are configured to permit flow of refrigerant sufficient to cool at least the surface 114A of the table 112. Refrigerant fluid, e.g., liquid refrigerant, enters the channel 118 through the entrance orifice 122A, moves through the channel 118, and exists through the exit orifice 122B.

In one embodiment, the table 112 further includes an insulation plate 124 coupled with the lower plate 116 and configured to provide insulation to the table 112. In one embodiment, the insulation plate 124 is foam insulation that is affixed, e.g., glued, to the lower plate 116. The lower plate 116 includes a number of orifices 126 configured to help to permit pressure relief so that if the system 100 does build up excessive pressure the pressure may be relieved via the orifices 126 in the lower plate 116.

In one embodiment, the table 112 may also include a drive shaft (not shown) operatively coupled along the central axis 113 of the table 112. The drive shaft may be operatively coupled with a drive motor (not shown) that is configured and operative to rotate the drive shaft causing the table 112 to rotate about the central axis 113, e.g. during processing of product mix disposed on the plate surface 114A.

As shown in FIGS. 1 and 3, at least a portion of a thermocouple assembly 128 passes through the lower plate 116 and is connected or fastened to the upper plate 114. A thermocouple 130 of the assembly 128 is disposed at a distance, e.g., within about 0.005 inches to about 0.01 inch, from the surface 114A of the upper plate 114. The thermocouple assembly 128 is part of the system 100 of the invention and is configured and operative to measure temperatures of the processing table 112 and/or its surface 114A. Alternatively, or additionally, the assembly 128 is part of a feedback loop for temperature adjustment and control of a food processing system or apparatus in which the system 100 is incorporated or integrated therewith.

In one embodiment, the thermocouple 130 transmits signals to a CPU or a controller 132, as shown in FIG. 1, that is part of the system 100 and/or is part of a food processing system or apparatus in which the system 100 is incorporated or integrated therewith. The CPU or controller 132 may include a sub-controller configured and dedicated to measure and help adjust and control temperatures of the table 112 and/or its surface 114A. In this case, the CPU or controller 132 may operatively couple with a system controller 138 to provide temperature adjustment and control, as well as adjustment and control of other operation variables and parameters of a food processing systems or apparatuses.

As shown in FIG. 1, the system 100 includes a chiller control 134 that is configured and operative to control flow of a refrigerant fluid into the refrigerant channel 118 of the table 112. The chiller control 134 receives one or more signals from the CPU or controller 132 that provide temperature adjustment and control instructions to the chiller control 134 to help to adjust and maintain temperatures of the table 112 and/or its surface 114A. Such instructions may relate to, for example, a duration, rate, or volume of flow of refrigerant fluid into the table 112. The instruction signals that the CPU or controller 132 transmits to the chiller control 134 may depend on the current or actual temperatures of the table 112 and/or its surface 114A that the thermocouple 130 measures and transmits to the CPU or controller 132. Alternatively, or additionally, the instruction signals that the CPU or controller 132 transmits to the chiller control 134 may depend on the current or actual temperatures of any food product ingredient(s) or intermediate(s) to be depositedon the table surface 114A for processing, as described below.

In one embodiment, the chiller control 134 may include a sub-controller that receives one or more signals from the CPU or controller 132, and that controls one or more valves or metering devices (not shown). The one or more valves or metering devices may operatively couple with the refrigerant fluid input lines 134A and 136A and return lines 134B and 136B to help to regulate flow of refrigerant fluid into the channel 118 of the table 112. The return lines 134B and 136B may circulate the refrigerant fluid exiting from the table 112 to a chiller, a refrigeration system, or an air conditioning system of a system or apparatus in which the system 100 of the invention is incorporated or integrated with. The fluid input lines 134A and 136A connect operatively to the entrance orifice 122A of the table 112 and the fluid return lines 134B and 136B operatively connect to the exit orifice 122B. The sub-controller of the chiller control 134 activates any of theses one or more valves or metering devices in response to signals received from the CPU or controller 138 and/or the system controller 138.

Alternatively, in another embodiment, the chiller control 134 does not include a sub controller. As such any of the one or more valves or metering devices would be activated from signals received from the CPU or controller and/or the system controller 138.

The system 100 also includes a thermocouple assembly 140 operatively coupled with one or more food product ingredient containers 144. Depending on the food product to be produced, certain containers 144 may hold, for example, base mixes, e.g., for making ice cream or frozen yoghurt, and a variety of flavorings, e.g., vanilla, chocolate, or strawberry, etc., etc., or other additives or ingredients that are typically combined to provide a product mix that is further processed at the table 112. In one embodiment, the thermocouple assembly 140 may include one or more thermocouples 142. Each thermocouple 142 may be configured and operative to measure temperatures of an ingredient or intermediate contained within its container 144 and to transmit signals to the CPU or controller 132 indicating temperatures of the ingredient or intermediate within its container 144. Similar to the thermocouple assembly 128 of the table 112, the thermocouple assembly 140 of the one or more containers 144 is part of the system 100 of the invention and is configured and operative to measure temperatures of the ingredients or intermediates in the containers 144.

The CPU or controller 132 includes memory (not shown) that stores a database, e.g., one or more look-up tables. In addition, the CPU or controller 132 memory stores a program algorithm configured and operative for processing signals that the CPU or controller 132 receives from the thermocouple assemblies 128 and 140, as well as, optionally, from the system controller 138. In addition, the program algorithm is configured and operative for providing instructions, in the form of computer readable medium, to the chiller control 134 and/or the system controller 138.

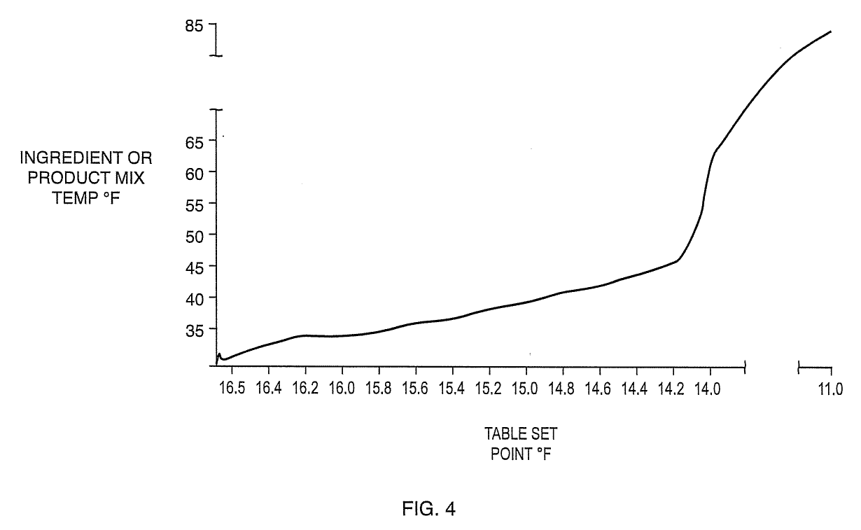

Referring to FIG. 4, and with further reference to FIG. 1, the CPU or controller 132 receives signals transmitted from the table thermocouple assembly 128 indicating the temperature(s) of the table 112 and/or its surface 114A. The invention adjusts dynamically the set point temperatures of the table 112 and/or its surface 114A via program instructions the CPU or controller 132 provides to the chiller control 134, which affects flow of refrigerant fluid into the channel 118 of the table 112.

In addition, in one embodiment, the CPU, or controller 132 may provide instructions to the chiller control 134 that affect temperatures of the refrigerant fluid circulating through the table 112. The CPU or controller 132 sends instructions related to adjustments of the table 112 set point temperatures depending on the temperatures of the food product ingredients that will be used to produce a particular food product. Depending on the actual or current temperatures of the table 112 and/or its surface 114A. The thermocouple assembly 140 provides one or more signals to the CPU or controller 132 indicating temperatures of the selected ingredient or intermediate. The CPU or controller 132 receives the signals and employs, for instance, one or more look-up tables to determine the appropriate or predetermined table 112 set point temperature(s) based on the temperature value(s) of the ingredient or intermediate that the received signal(s) represent.

The CPU or controller 132 also receives signals from the thermocouple assembly 128 that indicate the actual or current temperatures of the table 112 and/or its surface 114A. The program algorithm processes the set point temperature(s) and the actual or current temperature(s) and provides instructions that the CPU or controller 132 transmits to the chiller control 134 to adjust the table 112 to the predetermined set point temperatures. The adjusted table 112 temperatures will thereby help to accommodate or offset the temperatures of the food product ingredient, when such ingredient is disposed along the table surface 114A.

As shown in the graph of FIG. 4, the system 100 employs the program algorithm to adjust and control the table 112 temperatures (along X-axis) depending on temperatures (along Y-axis) of the food ingredient. In addition, the program algorithm can further adjust and control the table 112 temperatures depending on temperatures of the table 112 and/or its surface 114A, for example, during processing along the surface 114A of the selected ingredient or a product mix that includes the selected ingredient. The program algorithm may initially adjust temperatures of the table 112 and/or its surface 114 to predetermined set point temperatures in accordance with actual measured temperatures of the selected food product ingredient or intermediate. The CPU or controller 128 would provide program instructions for the temperature adjustment to the chiller control 134, and the chiller control 134 would operate to adjust the temperatures of the table 112 and/or its surface 114A to the predetermined set point temperatures. In this manner, the temperatures of the table 112 and/or its surface 114A help to accommodate or offset the temperatures of the selected food product ingredient. Once the system 100 reaches the predetermined set point temperatures, the CPU or controller 128 may continue to employ temperatures received from the thermocouple assembly 128 to continuously or periodically monitor and adjust the temperatures of the table 112 and/or its surface 114A during processing.

In one example, an ingredient, such as a base mix for an ice cream product, may be loaded into, and/or stored within, one of the containers 144 at an ambient temperature, such as about 85° F. For optimal processing, a system or apparatus may require the base mix at a lower temperature, such as about 40° F., in order to produce a certain ice cream product from the base mix that has certain required or desired properties or qualities. The system 100 of the invention helps to accommodate for the current elevated temperature of the base mix by lowering the temperatures of the table 112 and/or its surface 114A to predetermined set point temperatures. The predetermined set point temperatures will help to accommodate or offset the thermal load along the table surface 114A when the base mix, or a product mix containing the base mix, is deposited along the surface 114A for processing, as well as to adjust the base mix or product mix to optimal or preferred processing temperatures. In addition, during processing, the CPU or controller 128 may continue to monitor and adjust dynamically the temperatures of the table 112 and/or its surface 114A to help to accommodate decreases in temperatures of the base or product mix and increases in temperatures of the table 112 and/or its surface 114A.

In another embodiment, the system 100 of the invention alternatively or additionally may use temperatures of the product mix, such as, for example, a base mix and one or more flavorings and/or other additives, before such product mix deposits along the table surface 114A for processing. In this case, the base mix and the one or more additives, may be mixed and/or aerated in a mixing and/or aerating apparatus, such as the conduit assembly 120 described in the assignee's U.S. Publication No. 2006/0054614 A1, or the turbulence tube 110 described in the assignee's U.S. Publication No. 2006/0162347 A1, or the mixing tube 127 described in the assignee's U.S. Publication No. 2007/0251260 A1. One or more thermocouple assemblies may be associated with any of such mixing and/or aerating apparatus to help to adjust temperatures of the table 112 and/or its surface 114A, such that, the system 100 may accommodate or offset any thermal load along the surface 114A that results from depositing the product mix along the table surface 114A.

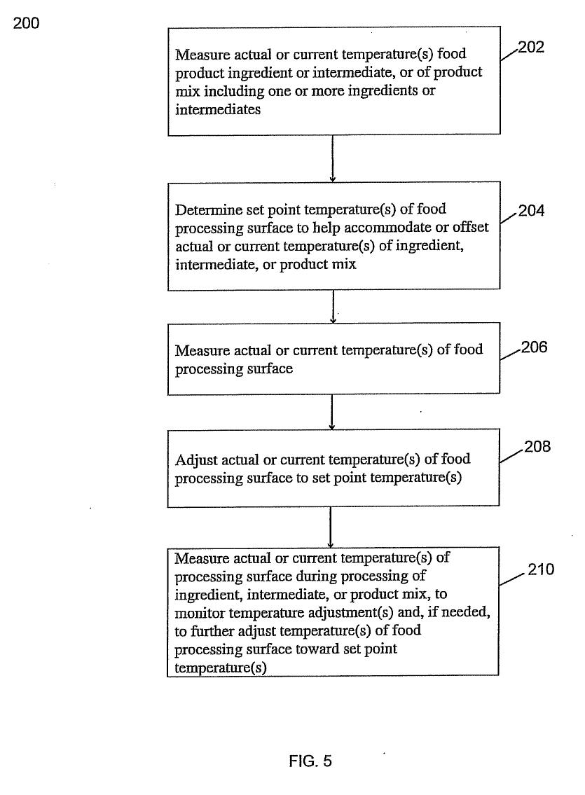

In operation, referring to FIG. 5, and with further reference to FIGS. 1-3, a flow diagram illustrates another aspect of the invention providing a method 200. The method 200 adjusts and controls temperatures, e.g., set point temperatures, of the processing table 112 and/or its surface 114A to help to accommodate or offset temperatures of one or more food product ingredients or intermediates, and/or a product mix including one or more ingredients or intermediates. In addition, the method 200 helps to adjust and control temperatures of food product ingredients or intermediates, and/or a product mix including one or more ingredients or intermediates, during processing. The methods 200 shown in the flow diagrams illustrated in FIGS. 5 and 6 are exemplary only and not limiting. The methods 200 may be altered, e.g., by having stages added, removed, and/or rearranged.

At stage 202, the thermocouple assembly 140 measures and transmits signals to the CPU or controller 132 representing actual or current temperatures of one or more food product ingredients or intermediates, e.g., a base mix and/or flavoring, and/or a product mix including one or more ingredients or intermediates.

At stage 204, the CPU or controller 132 employs one or more look-up tables to determine set point temperatures of the table 112, and/or its surface 114A, to help to offset the current or actual temperatures of the food product ingredient(s) or intermediate(s). At stage 206, the thermocouple assembly 128 measures and transmits signals representing actual or current temperatures of the table 112, and/or its surface, to the CPU or controller 132.

At stage 208, the program algorithm provides instructions that the CPU or controller 132 transmits to the chiller control 134 to adjust flow of refrigerant fluid into the table 112 to help to adjust the actual or current temperatures of the table 112, and/or its surface 114A, toward the predetermined set point temperatures.

Optionally, as illustrated in FIG. 6, at stage 210, during processing of the one or more ingredients or intermediates, and/or during processing of the product mix, along the table surface 114A, the thermocouple assembly 128 transmits actual or current temperatures of the table 112, and/or its surface 114A, to the CPU or controller 134 in order that the CPU or controller 134 may monitor the progress of temperature adjustment of the table surface 114A and, if necessary, to further adjust temperatures of the table surface 114A during processing.

The system 100 of the invention is advantageous for use in an automatic food processing and/or dispensing system or apparatus whereby food product ingredients or intermediates are stored within, or loaded into, the system or apparatus at temperatures other than temperatures required or desired for producing a food product. The system 100 of the invention thereby helps to avoid or reduce the downtime of such a system or apparatus that may be required in order to adjust temperatures of food product ingredients and intermediates for processing. The system 100 of the invention is advantageous in that it helps to ensure that a system or apparatus consistently and efficiently produces food products with certain required or desired properties or qualities.

Having thus described at least one illustrative embodiment of the inventions, various alterations, substitutions, modifications, and improvements in form and detail will readily occur to those skilled in the art without departing from the scope of the inventions. Such alterations, substitutions, modifications, and improvements are intended to be within the scope and spirit of the inventions. Other aspects, functions, capabilities, and advantages of the inventions are also within their scope. Accordingly, the foregoing description is by way of example only and is not intended as limiting.

In addition, in describing aspects of the invention, specific terminology is used for the sake of clarity. For purposes of description, each specific term is intended to at least include all technical and functional equivalents that operate in a similar manner to accomplish a similar purpose. In some instances where a particular aspect of the invention includes a plurality of system elements or method steps, those elements or steps may be replaced with a single element or step; likewise, a single element or step may be replaced with a plurality of elements or steps that serve the same purpose. Further, where parameters for various properties are specified herein for aspects of the inventions, those parameters can be adjusted or rounded-off to approximations thereof within the scope of the invention, unless otherwise specified.

What is claimed is:

1. A method for adjusting and controlling the temperature of a food processing and dispensing device, the method comprising
measuring, by a first thermocouple assembly, the actual temperature of one or more food product ingredients;
transmitting one or more signals to a CPU, the signals representing the actual temperature of the one or more food product ingredients measured by the first thermocouple assembly;
employing one or more look-up tables within the CPU to determine a set point temperature of a food processing surface;
measuring, by a second thermocouple assembly, the actual temperature of the food processing surface;
transmitting one or more signals to the CPU representing the actual temperature of the food processing surface; and
adjusting dynamically, the temperature of the food processing surface to the is determined set point temperature.
